(19) United States
(12) Patent Application Publication
(10) Pub. No.: US 2005/0085922 A1
(43) Pub. Date: Apr. 21, 2005

(54) Shaped filler for implantation into a bone void and methods of manufacture and use thereof

(76) Inventors: Ben R. Shappley, Germantown, TN (US); Thomas J. Bradbury, Yardley, PA (US); Peter A. Materna, Metuchen, NJ (US); Timothy J. Pryor, Yardley, PA (US); John Blum, Somerset, NJ (US); Ali Cem Recber, Piscataway, NJ (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 10/966,113

(22) Filed: Oct. 18, 2004

Related U.S. Application Data
(60) Provisional application No. 60/512,498, filed on Oct. 17, 2003.
(60) Provisional application No. 60/512,414, filed on Oct. 17, 2003.
(60) Provisional application No. 60/577,736, filed on Jun. 7, 2004.

(51) Int. Cl. A61F 2/28

(52) U.S. Cl. 623/23.5; 623/23.63; 623/23.56

(57) ABSTRACT

The invention is directed to shaped bone void filler pieces having defined porosity. In embodiments of the invention, the shaped bone void filler pieces are presented substantially as wedges, wafers, and axisymmetric bone void filler pieces. The bone void filler pieces further comprise surface and internal features such as recesses, channels, and/or voids. The bone void filler pieces optionally comprise demineralized bone matrix. The invention further is directed to methods of making and methods of using the bone void filler pieces. In another embodiment of the invention, the bone void filler pieces are produced using three dimensional printing methods. In yet another embodiment of the invention, the bone void filler pieces are manufactured with selected porogens integrated therein, which optionally are decomposed following production through a heat-mediated decomposition process, resulting in voids in the bone void filler spaces previously occupied by the porogen(s).

### CROSS-REFERENCE TO RELATED APPLICATIONS

This application claims the benefit of U.S. Provisional Application No. 60/512,498, filed Oct. 17, 2003; U.S. Provisional Application No. 60/512,414, filed Oct. 17, 2003; and U.S. Provisional Application No. 60/577,736, filed Jun. 7, 2004, the disclosures of each of which are herein incorporated by reference in their entireties.

### BACKGROUND OF THE INVENTION

Voids are surgically created in bones for a variety of reasons, including donation of bone for use at another site, treatment of cancer or bone necrosis, and repair of traumatic injury or congenital conditions. The materials known to be used for filler in bone voids include collagen, allograft, bone chips obtained from the patient during surgery, demineralized bone matrix, and ceramic materials in the form of granules. Ceramic materials are of interest at least because of their ready availability and the avoidance of possible disease transmission to the patient. Members of the calcium phosphate family have chemical similarity to the inorganic component of natural bone, and tricalcium phosphate is of particular interest in this field due to its resorbability.

When a piece of bone is surgically removed, such as at a bone donor site, the void has typically been filled by any of several types of filler material. In some instances, a putty-like material has been used, and in other instances loose granular materials have been used. However, both putty-like materials and loose granular materials have the potential to migrate after surgery. In still other instances, the bone void has been filled by a block filler piece, such as a block of synthetic material, which has been manufactured to an oversized standard shape that is carved during surgery to fit the bone void. Filler pieces and materials which are in current use for filling bone donor sites have often not resulted in ingrown bone of the same quality as the removed bone, sometimes resulting in adhesions between regrown bone and healed adjacent soft tissue, with resulting acute, idiopathic or chronic pain to the patient.

Where the bone void filler is autograft, current surgical techniques for harvesting bone for autografts results in bone bleeding to an extent that the bleeding sometimes obscures observation of the site of the cut and hinders fitting of the filler piece to the bone void.

In general, a rigid bone void filler piece is useful for encouraging bone ingrowth if it includes patterning on those surfaces that touch native bone, and also if the bone void filler piece has channels through it. However, if the surface of a filler piece is cut and shaped to fit during surgery, detailed surface patterns from the original manufacture might be removed during cutting and shaping and therefore may not remain after cutting and shaping.

Ceramic materials are of interest in bone substitutes at least because of their ready availability and the avoidance of possible disease transmission from a donor to a patient. Members of the calcium phosphate family are of interest and have chemical similarity to the inorganic component of natural bone, and tricalcium phosphate is of particular interest due to its resorbability.

Accordingly, it is desirable to provide a bone void filler piece whose pre-manufactured surface, which may contain surface patterns and/or channels, is substantially the final surface that adjoins native bone when installed. There also remains a need for bone void fillers in shapes specific to fill axisymmetric or wedge-shaped bone voids, especially in a tightly-fitting manner.

It is desirable to provide a bone void filler piece having a geometry of internal channels that is conducive to ingrowth of natural bone. It is further desirable to provide a bone void filler piece which wicks blood, plasma and other bodily fluids so as to promote ingrowth of natural bone.

It is also desirable to provide a bone void filler piece that is suitable to conform to a bone void suitably to stop or at least reduce the flow of blood from the cut bone. It is desirable to provide a bone void filler piece that is resorbable so as to eventually be completely replaced by natural bone.

It is desirable to provide a kit that includes cutting tools and/or templates or other items needed to create a bone void that matches the shape of the already-manufactured filler piece, or includes other surgical items.

### BRIEF SUMMARY OF THE INVENTION

The invention is directed to shaped bone void filler pieces having defined porosity. In embodiments of the invention, the shaped bone void filler pieces are presented substantially as wedges, wafers, and axisymmetric bone void filler pieces. The bone void filler pieces further comprise surface and internal features such as recesses, channels, and/or voids. The bone void filler pieces optionally comprise demineralized bone matrix. The invention further is directed to methods of making and methods of using the bone void filler pieces. In another embodiment of the invention, the bone void filler pieces are produced using three dimensional printing methods. In yet another embodiment of the invention, the bone void filler pieces are manufactured with selected porogens integrated therein, which optionally are decomposed following production through a heat-mediated decomposition process, resulting in voids in the bone void filler spaces previously occupied by the porogen(s).

### BRIEF DESCRIPTION OF THE DRAWINGS

FIG. 1 shows the shape of the filler piece as being prismatic.

FIG. 2 shows features which in combination may make the wedge-shaped filler piece suitable to stuff into a bone void, for example to reduce or stop the flow of blood from a cut bone.

FIG. 3 shows one embodiment of the wafer-shaped bone void filler piece, having recesses or channels therethrough.

FIGS. 4A-4B show embodiments of the wafer-shaped bone void filler piece, having recesses or channels therethrough.

FIG. 5 shows one embodiment of an axisymmetric bone void filler piece comprising, or alternatively consisting of, a cylindrical bone void filler further comprising internal dead-end channels or surface indentations.

FIG. 6 shows one embodiment of an axisymmetric bone void filler piece comprising, or alternatively consisting of, a cylindrical bone void filler piece further comprising surface indentations and through-channels, and further comprising a chamfer suitable for guiding the bone void filler into place.

FIG. 7 shows an axisymmetric bone void filler produced in accordance with the embodiment described for FIG. 6.

FIG. 8A shows a bone void filler piece that is frusto-conical for all of its length, having an overall apex angle as indicated. FIG. 8B shows a bone void filler piece that is frusto-conical for the majority of its length, having an overall apex angle as indicated, and further having a chamfer at the narrower end.

FIG. 9 shows an SEM photograph showing typical microstructure including porosity and pore sizes as well as the interconnected nature of the porosity of the axisymmetric bone void filler pieces.

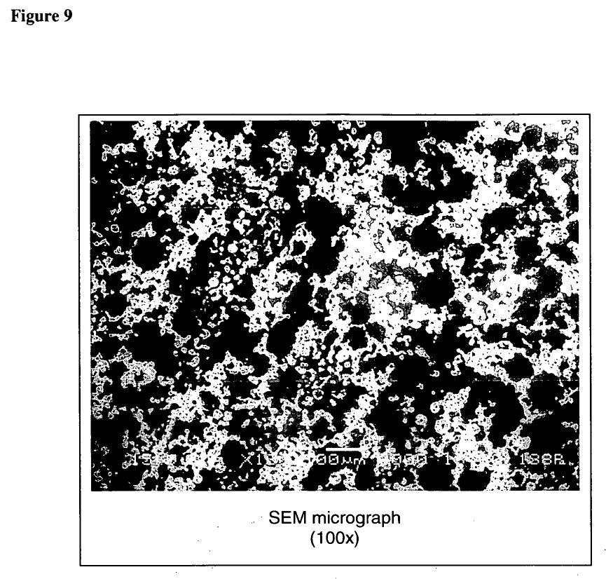

FIG. 10 shows an apparatus suitable for performing three dimensional printing.

FIG. 11 shows a general schematic of a 3DP manufacturing process.

### DETAILED DESCRIPTION OF PREFERRED EMBODIMENTS

The invention includes bone void filler pieces of shapes suitable for implantation in a bone void that was surgically created, or otherwise resulting from other conditions such as disease or traumatic injury. The filler pieces may be suitable for use in a void in the crest of the ileum, which may be used as a bone donor site.

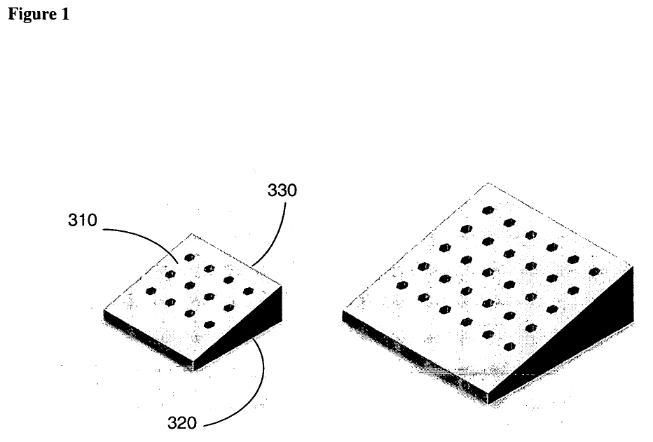

In one embodiment of the invention, the shape of the filler piece may be a shape that is substantially a wedge or a truncated wedge. The wedge or truncated wedge may be defined by a first generally flat surface and a second generally flat surface that is angled with respect to the first generally flat surface. The two surfaces may be angled with respect to each other by an angle of about 15 degrees of less, or more generally, an angle of less than about 30 degrees. The filler piece may also comprise a base surface connecting the two generally flat surfaces at the larger end of the filler piece. FIG. 1 shows the shape of the filler piece as being prismatic, in the sense that a base shape, which is substantially a trapezoid, is extended into the third dimension along a direction which is substantially perpendicular to the plane of the base shape.

The wedge embodiment of the invention may have edges that are rounded or otherwise modified. Similarly, the surfaces of the bone void filler wedge may have planar surfaces that are flat planes, or alternatively the surfaces need not be exactly flat. More generally, the wedge filler piece may have a wider end and a narrower end and, connecting the two ends, a continuous surface. The bone-contacting surfaces may be generally mutually opposed and have local tangents which, when brought out to intersect in the direction of the narrow end of the filler piece, form a small angle. The angle (which is a total included angle) may be, for example, less than about 30 degrees. In general, it is not necessary for all of the tangents to intersect at a single line or a single point. In general, it is not necessary for the angle of intersection of the tangents to be the same everywhere around the filler piece or for all points on the bone-contacting surfaces of the filler piece.

The filler piece may have a defined local surface geometry in at least some surfaces that are intended to adjoin native bone when the filler piece is implanted in a patient, such as the two generally flat surfaces. The defined surface geometry of the wedge bone void filler may comprise, or alternatively may consist of, surface recesses such as dimples, depressions, grooves, and the like suitable to promote the ingrowth of natural bone. The defined surface geometry may comprise channels extending through the filler piece from the first generally flat surface to the second generally flat surface, or alternatively extending in any other geometry including dead-ended channels, suitable to promote the ingrowth of natural bone.

Channels may be of any cross-sectional shape including round, rectangular and other cross-sectional shapes. Such surface recesses or channels may have a smallest dimension, along a surface of the filler piece, which is in the range from about 50 micrometers to about 3000 micrometers. The filler piece may have both channels and surface recesses, in any combination, on any surface. For example, it is known that for physiological reasons, there is a maximum distance typically of about 2 millimeters through which nutrients and waste products can diffuse between a cell and the nearest blood vessel. In other words, almost all cells in the human body are generally less than that distance from some blood vessel. In another embodiment of the invention, the bone void filler piece may be designed so that every point in the filler piece is within about 2 millimeters of a channel, surface recess or similar feature. It is assumed that the channels and surface recesses in the filler piece may become pathways of vascularization, as well as serving as pathways for early progression of tissue ingrowth into the filler piece.

In one embodiment of the invention, the bone void filler piece of the invention comprises, or alternatively consists of, a wedge-shaped bone void filler piece having an average pore size of about 60 micrometers, a range of pore size from about 7 micrometers to about 900 micrometers, and an overall porosity of about 53% to about 70%.

More generally, the wedge-shaped bone void filler piece of the invention may have an overall porosity of about 40% to about 70%, a range of pore size from about 1 micrometer to about 300 micrometers, and recessed features having cross-dimensions in the range of about 50 micrometers to about 3000 micrometers.

The bone void filler wedge piece may be installed into the bone void without requiring any substantial modification of any surface of the filler piece adjoining natural bone. In this case, the as-manufactured surface of the filler piece may touch natural bone. Alternatively, it is possible that the filler piece may require removal of material from the piece before installation, such as to improve fit, for example.

In an embodiment of the invention, the bone void filler wedge piece as illustrated in FIG. 1 comprises channels transiting through the wedge-shaped piece from the first generally flat surface [310] to the second generally flat surface [320]. The wedge further includes a base surface [330] connecting the two generally flat surfaces at the larger end of the filler piece. In this embodiment, a substantial amount of material may be removed from the surface of the piece without destruction of the features, i.e., the channels would still be apparent even after such removal of material.

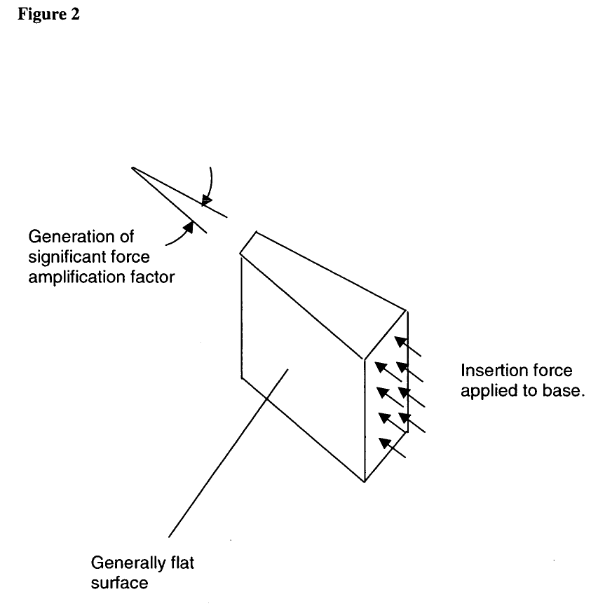

The wedge-shaped bone void filler piece may comprise a combination of features allowing the wedge piece to be used to suitably “stuff” a bone void to reduce or stop the flow of blood from freshly cut bone. This combination of features includes the close-fitting nature of the wedge piece with respect to the void in the bone. This may be determined by the coordination of the filler piece, templates, cutting tools, and by surgical technique. FIG. 2 displays features which in combination may make the wedge-shaped filler piece suitable to stuff into a bone void, for example to reduce or stop the flow of blood from a cut bone.

Another feature of the wedge piece is that the filler piece may be made of a material that is suitable for local crushing under the application of a specified local pressure. For example, the material of the filler piece may be softer than the adjacent native bone so that the filler piece can crush at points of concentrated loading within the region of interaction between the filler piece and the cut surfaces of native bone. This crushability can serve to accommodate local irregularities or dimensional mismatches between the filler piece and the bone void, through localized crushing of the filler piece, so as to provide a tighter fit compared to that achieved by the undeformed filler piece.

An additional feature of the wedge-shaped filler piece is that the described shape of a wedge or truncated wedge, which may be useful for receiving an insertion force for introducing the wedge-shaped filler piece into the similarly-shaped bone void, may result in a force amplification factor that generates relatively large forces between the filler piece and the adjacent native bone. Such force may be useful for causing localized crushing of the filler piece material and also for maintaining the filler piece in contact with the adjacent native bone by friction. The force amplification factor associated with the wedge geometry increases as the apex angle of the wedge or truncated wedge decreases. For example, the total included angle of the wedge-shaped piece may be somewhat small, such as less than about 15 degrees, or more generally, less than about 30 degrees. For geometries that comprise imperfect wedges, the angle between tangents from opposing sides may also be less than about 30 degrees.

Another additional feature of the wedge-shaped filler piece useful for stuffing a bone void is that the filler piece may be designed so that the insertion force suitable to create localized crushing at the bone-facing surfaces may be applied to an external surface of the filler piece without creating an excessive local pressure at that external surface so as to cause local crushing at said external surface. In a wedge-shaped filler piece, the exposed external surface refers to the base of the wedge. This may be achieved through use of an insertion (pushing) tool which substantially conforms to the exposed external surface of the filler piece, and which also may contact a large fraction of the exposed external surface of the filler piece. It is possible that both the insertion tool and the external surface of the filler piece may be substantially flat. The described external geometry of the filler piece may allow the application of insertion forces to the filler piece without causing localized crushing of the external surface at the site of application of the insertion force, while concurrently resulting in localized crushing at the appropriate bone-facing locations on the filler piece.

These attributes and features, in combination, may allow the wedge-shaped filler piece to be used to reduce or stop the flow of blood from the cut bone. Even if this combination is not used for reducing or stopping blood flow, it is still useful for securing the wedge-shaped filler piece in the bone void by press-fit friction.

In another embodiment of the invention, the bone void filler piece takes the shape of a wafer. The wafer-shaped bone void filler piece may have two surfaces which are approximately parallel to each other and are separated by an approximately uniform thickness in the dimension transverse to the approximately parallel surfaces. The thickness of the wafer-shaped piece may be substantially smaller than its dimensions in the other directions. As far as the overall external shape, the wafer may be round, although this is optional.

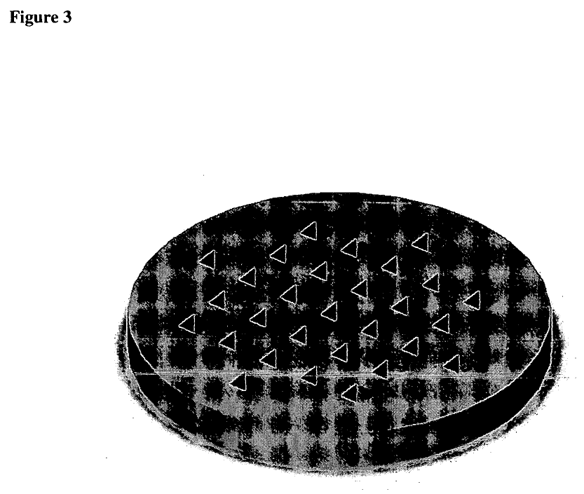

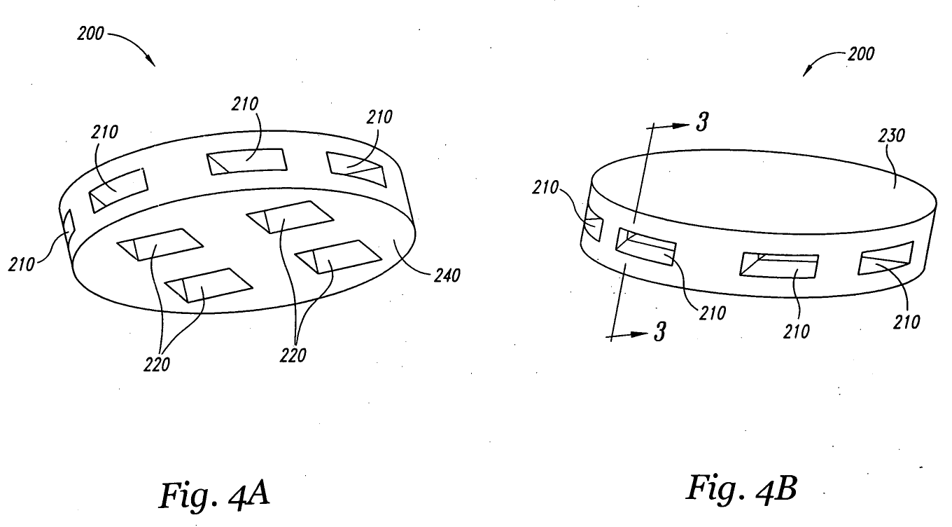

The wafer-shaped piece may further contain surface features such as dimples, depressions, or through-passageways, any of which may be conducive to the ingrowth of natural bone. These features, such as dimples or depressions, may be located in the surfaces which are about parallel to each other. Through-passageways may be of any cross-section including round, rectangular or other cross-sectional shapes. Such surface recesses or channels may have a smallest dimension, along a surface of the wafer, which is in the range from about 50 micrometers to about 500 micrometers. FIG. 3 and FIGS. 4A and 4B show embodiments of the wafer-shaped bone void filler pieces, having recesses or channels therethrough. FIGS. 4A and 4B display a biostructure of cylindrical or wafer exterior geometry comprising channels or surface recesses in two different coplanar directions, intersecting each other in horizontal and vertical directions. The horizontal and vertical channels in the present embodiment may be approximately 1.35 mm in height and width. The vertical channels [220] are shown as also intersecting the horizontal channels [210] at places where the various horizontal channels intersect each other. A top region [230] of the illustrated embodiments contains no surface features. A bottom region [240] includes vertical channels [220] that extend through the filler piece and may terminate prior to intersecting with the horizontal channels [230], at the intersection of the horizontal channels [230], or at some point beyond the intersection of the horizontal channels. Furthermore, each of the vertical channels [220] may terminate at a point independent of an adjacent channel. In alternative embodiments, the vertical and horizontal channels may be angled, non-linear, or have varying cross-sectional dimension.

In another embodiment of the invention, the bone void filler piece of the invention comprises, or alternatively consists of, a wafer-shaped bone void filler piece having an average pore size of about 60 micrometers, a range of pore size from about 7 micrometers to about 900 micrometers, and an overall porosity of about 53% to about 70%.

More generally, the wafer-shaped bone void filler piece of the invention may have an overall porosity of about 20% to about 45%, or an overall porosity of about 40% to about 70%, a range of pore size from about 8 micrometers to about 20 micrometers, or alternatively a range of pore size from about 1 micrometer to about 300 micrometers, and recessed features having cross-dimensions in the range of about 50 micrometers to about 500 micrometers.

The wafer-shaped piece may comprise any diameter suitable to fill a bone void, for example between two bone surfaces. In certain embodiments of the invention, the wafer-shaped piece has dimensions of 10 millimeters diameter by 3 millimeters axial dimension, 15 millimeters diameter by 5 millimeters axial dimension, or 20 millimeters diameter by 7 millimeters axial dimension.

The wafer-shaped piece may additionally have features which are helpful for gripping and lifting the wafer. Such features may include, but are not limited to, recesses, flat surfaces or perforations, which may cooperate with a tool such as tweezers. Such features may also be useful geometries for encouraging the ingrowth of native bone tissue.

In an additional embodiment of the invention, the bone void filler piece comprises, or alternatively consists of, an axisymmetric overall external shape. More specifically, the bone void filler may be either cylindrical or frusto-conical, either for about the entire length along the axis of symmetry, or at least a majority thereof. In the latter case, it is further possible that the piece may include aminority of length along the axis of symmetry comprising a chamfer or other reduced-diameter smooth, generally axisymmetric, shape suitable to help guide the piece into place.

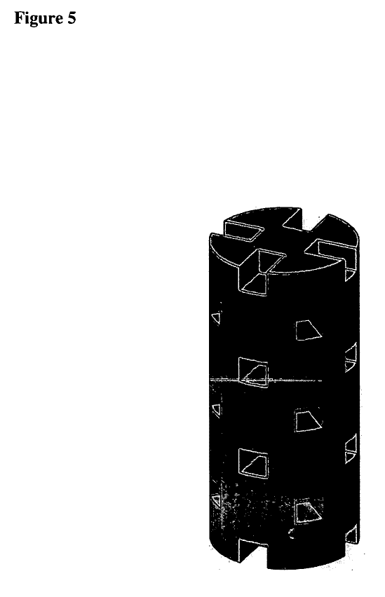

In one embodiment of the invention as provided in FIG. 5, the axisymmetric bone void filler piece comprises, or alternatively consists of, a cylindrical bone void filler further comprising internal dead-end channels or surface indentations. The features at the ends of this embodiment of bone void filler piece are suitable to engage tooling (such as tooling for handling the bone void filler piece or for inserting the bone void filler into a surgical site). In this embodiment, features are illustrated as surface recesses which are dead-ends and do not intersect other surface recesses. This may be accomplished in part by placing recesses at selected places that are staggered along the axial direction of the generally cylindrical geometry. In this embodiment the surface recesses are distributed in a symmetric pattern, although this is not necessary. In other embodiments, it is possible that the bone void filler could have features which are channels completely transiting through the bone void filler, or recesses which intersect with other recesses.

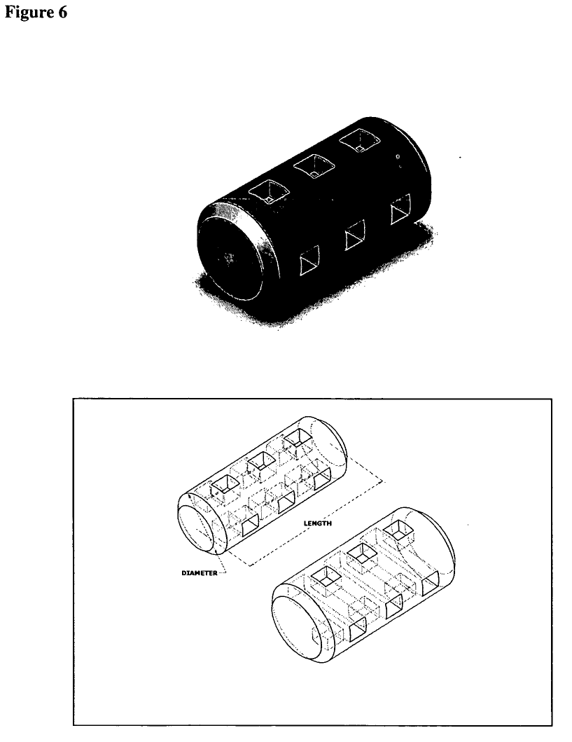

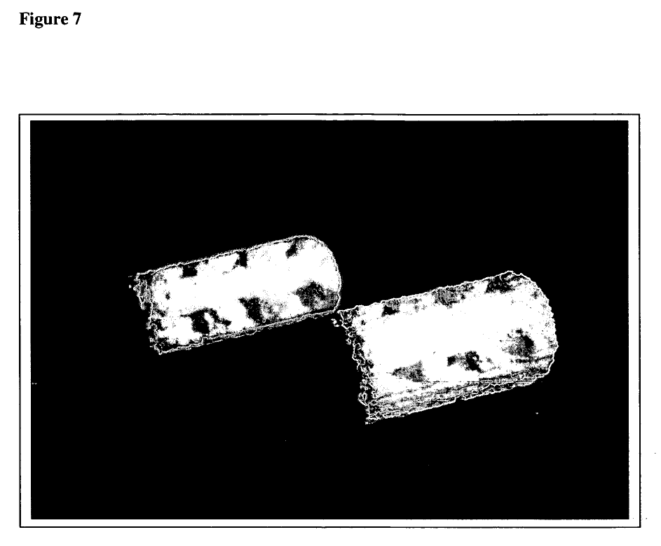

In another embodiment of the invention as provided in FIG. 6, the axisymmetric bone void filler piece comprises, or alternatively consists of, a cylindrical bone void filler piece further comprising surface indentations and through-channels, and further comprising a chamfer suitable for guiding the bone void filler into place. FIG. 7 shows an axisymmetric bone void filler produced in accordance with this embodiment.

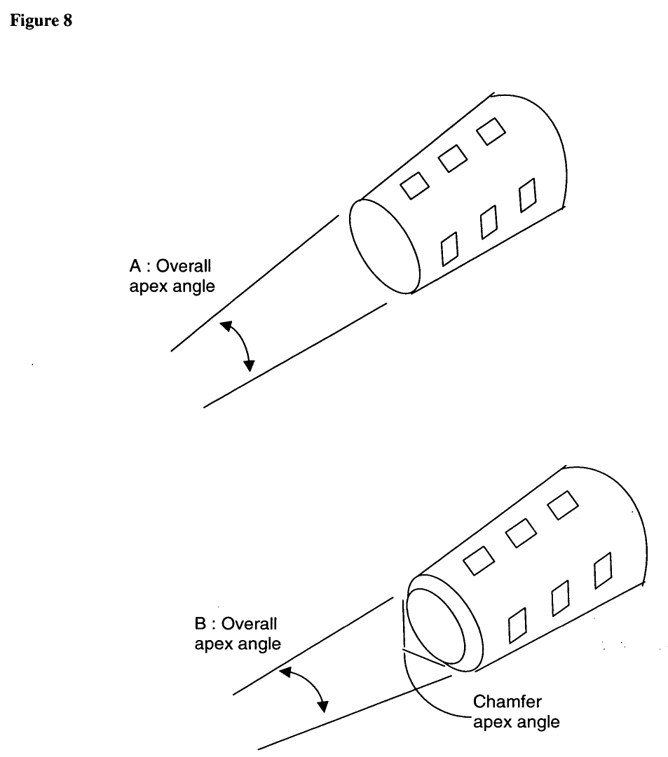

In yet another embodiment of the invention as provided in FIG. 8, the axisymmetric bone void filler piece comprises, or alternatively consists of, a bone void filler piece which is frusto-conical. In FIG. 8A, the bone void filler piece is frusto-conical for all of its length, having an overall apex angle as indicated. In FIG. 8B, the bone void filler piece is frusto-conical for the majority of its length, having an overall apex angle as indicated, and further having a chamfer at the narrower end. The chamfer may also be frusto-conical having its own chamfer apex angle (also indicated), with the chamfer apex angle being larger than the overall apex angle. Any such bone void filler piece may further comprise internal channels and surface recesses.

In another embodiment of the invention, the bone void filler piece of the invention comprises, or alternatively consists of, an axisymmetric bone void filler piece having an average pore size of about 60 micrometers, a range of pore size from about 7 micrometers to about 900 micrometers, and an overall porosity of about 53% to about 70%.

More generally, the wedge-shaped bone void filler piece of the invention may have an overall porosity of about 20% to about 50%, or alternatively an overall porosity of about 40% to about 70%, a range of pore size from about 1 micrometer to about 300 micrometers, from about 60 micrometers to about 90 micrometers, or from about 7 micrometers to about 1000 micrometers, and recessed features having cross-dimensions in the range of about 500 micrometers to about 3000 micrometers.

In any embodiment of the axisymmetric bone void filler piece having a chamfer or curved transition, the chamfer or curved transition may be substantially axisymmetric around an axis which substantially coincides with the axis of the bone void filler itself.

The axisymmetric bone void filler piece may have dimensions of an overall length of about 20.4 millimeters, and an outside diameter of about 8 millimeters to about 10.3 millimeters.

The axisymmetric bone void filler piece may have a geometry which is suitable for sliding, by a translational motion, into the bone void. This mode of insertion into the bone void is possible when the bone void is of a generally cylindrical geometry, or alternatively when the bone void is of a generally tapered shape such as conical or frusto-conical. In the event that the bone void and the bone void filler piece are non-axisymmetric, a limited number of positions of the bone void filler inside the bone void are possible. If, however, the bone void and the bone void filler piece are axisymmetric, the bone void filler could occupy any of many rotational angles.

Either the axisymmetric bone void filler piece or the bone void itself, or both, may comprise features of a helical nature such that at least one helical feature cooperates with another helical feature allowing the axisymmetric bone void filler piece to be inserted into the bone void with a combination of rotational and translational motion, such as being threaded into place inside the bone void. If the bone void filler is designed for installation by a combination of translational and rotational motion, it may have a geometry of a tapered screw.

The axisymmetric bone void filler piece may have any of a variety of features which act to retain the axisymmetric bone void filler piece inside the bone void, and create force between the axisymmetric bone void filler piece and the bone void. The existence of force by which the axisymmetric bone void filler piece bears against the bone void can be useful to prevent post-operative migration of the axisymmetric bone void filler piece from the bone void, and to improve the bone ingrowth process. These features and techniques may be used alone or in combination, depending on the surgical procedure used to install the bone void filler.

In another embodiment of the invention, the axisymmetric bone void filler piece is retained in a bone void by friction. The exterior of the axisymmetric bone void filler piece may have a close fit or a dimensional interference with a corresponding portion of the bone void. The close fit or dimensional interference may occur either with untapered shapes or with tapered shapes. The axisymmetric bone void filler piece may be manufactured with a taper (such as a frusto-conical shape), which may optionally correspond to a taper in the bone void.

A substantial portion of the axisymmetric bone void filler piece, or alternatively an exterior feature present on the surface thereof, is involved in the fit and/or interference of the axisymmetric bone void filler piece with the bone void. For example, features such as knurling, ribs or protrusions may be incorporated into the design of the axisymmetric bone void filler piece, such that only those features have close fit or interference. It is further possible that the axisymmetric bone void filler piece may be designed so that it, or appropriate features on the surface thereof, are capable of crushing upon installation. This feature could accommodate a wider range of tolerances for the manufacture of the axisymmetric bone void filler piece than is possible without planned crushing features. The entire bone contacting surface, or alternatively selected features or portions thereof, may be designed for crushing on implantation. Sufficient friction forces may be developed to keep the axisymmetric bone void filler piece in place within the bone void on implantation.

It is also possible that surgical procedures for installing the axisymmetric bone void filler piece may involve problems of limited access and/or poor visibility at the surgical site of implantation. Therefore, it is desirable to include in one embodiment of the design guiding features suitable to assist in the implantation of the axisymmetric bone void filler piece within the bone void. One such guiding feature which the axisymmetric bone void filler piece may have is a chamfer or taper, whereby the leading edge of the axisymmetric bone void filler piece has a loose fit within the bone void suitable for guiding the trailing portions of the axisymmetric bone void filler piece into the bone void. Insertion of the axisymmetric bone void filler piece into the bone void may be more difficult to achieve in the absence of a taper or guiding feature. One specific example of a guiding feature is the fit of a chamfer (localized frusto-conical portion) of an axisymmetric bone void filler piece into a bone void having a cylindrical or conical interior shape. It is also possible to use an axisymmetric transition shape whose cross-section is a smooth curve.

The axisymmetric bone void filler piece may optionally further comprise a carrying feature suitable to allow the axisymmetric bone void filler piece to be gripped or carried at the time the filler piece is inserted into the bone void. Such a feature may cooperate with an appropriate tool. Such a feature may, for example, include recessed or flat surfaces which may cooperate with a tool such as tweezers. Such a feature may, for example, be a hole which extends some distance into the axisymmetric bone void filler piece. The hole may have a non-round cross-section and may cooperate with a similarly-shaped tool. Such a shape and corresponding tool provides control over the angular orientation of the axisymmetric bone void filler piece during implantation, and could be used to rotate the axisymmetric bone void filler piece if the implantation required any rotational motion.

The axisymmetric bone void filler piece may have a plurality of recessed or internal features at any one or more of its surfaces. Recessed or internal features may be considered to be any form of material feature such as a channel through the piece or a recess in the piece which occupies aminority of the surface or internal area. Recessed or internal features encompass dead-ended recesses or channels which go through the bone void filler and exit at a surface of the bone void filler. In general, any surface or combination of surfaces may be provided with such recessed or internal features. The distribution of such features can in general be of any pattern on any surface or combination of surfaces of the axisymmetric bone void filler piece. Such surface recesses or channels may have a smallest dimension, along a surface of the axisymmetric bone void filler piece, which is in the range from about 500 micrometers to about 3000 micrometers.

It is further possible, assuming that the bone void has angular features, that the locations of the surface recesses or channels in the axisymmetric bone void filler piece could be coordinated with the location of the angular features in the bone void. The surface recesses or channels may, for example, be helpful for establishing vascularization in support of new tissue growth. Ensuring such alignment is possible in the case of rotational symmetry, if at least some non-symmetric feature is provided in either the bone void or the bone void filler to define the relative angular orientation of the bone void and the axisymmetric bone void filler piece.

Alternatively, it is possible that the recesses or internal features could be dead-end recesses. The axisymmetric bone void filler piece may include dead-end recesses having depth up to or exceeding the radius of the axisymmetric bone void filler piece, while also providing surface recesses having a depth not exceeding the radius of the axisymmetric bone void filler piece.

The axisymmetric bone void filler piece may also comprise isolated high spots or ribs, which may be suitable to be crushed during installation of the filler piece. The axisymmetric bone void filler piece may have a carrying feature suitable to allow the filler piece to be gripped or carried by a carrying tool.

The axisymmetric bone void filler piece may be made of particles of a matrix material that are partially joined directly to each other. The filler piece may be porous, having a specified porosity and a pore size distribution. One possible set of porosity and pore size distribution is described in “Bone void filler and method of manufacture,” U.S. Provisional Patent Application No. 60/466,884, filed Apr. 30, 2003, or U.S. patent application Ser. No. 10/837,541, filed Apr. 30, 2004, the disclosure of each of which is herein incorporated by reference, as having an average peak in the pore size distribution of about 60 micrometers. Another possible porosity and pore size distribution is described in U.S. patent application Ser. No. 10/122,129, filed Apr. 12, 2002, the disclosure of which is herein incorporated by reference, as having an average peak in the pore size distribution at about 8 to 20 micrometers. Overall porosities for both of these average pore size distributions may be in the range from about 40% to about 70%. For demineralized bone matrix and polymers, the average pore sizes may be larger, such as in the tens or hundreds of microns. Another possible set of properties is a pore size distribution having an average pore size of about 60 micrometers to about 90 micrometers, an actual range of pore sizes from about 7 micrometers to about 1000 micrometers, and an overall porosity in the range of from about 50% to about 70%. In the case of an axisymmetric bone void filler piece comprising demineralized bone matrix, the overall porosity may be in the range of from about 50% to about 60%. In the case of a bone void filler comprising polymer, the overall porosity may be in the range of from about 40% to about 70%. For demineralized bone matrix and polymer, the pore sizes may be in the tens or hundreds of microns. These are not exact requirements, however.

The axisymmetric bone void filler piece may be made of a material and have a geometry that is suitable to promote wicking of bodily fluids into the filler piece. Wicking of bodily fluids may be advantageous in promoting ingrowth of natural bone. For example, the porosity and pore size parameters described herein are suitable to promote wicking of bodily fluids that are not drastically different from water in their physical properties.

The axisymmetric bone void filler piece may also be of a hardness such that it can easily be carved, abraded or cut with a knife, or otherwise have material removed from it during surgery. For example, the filler piece may have properties enabling cutting and abradability, so that the piece resembles the properties of common chalk or mineral chalk. Non-limiting examples of surfaces of the filler piece which might be subject to shaping during surgery include the first and second large generally flat surfaces, and the base.

The axisymmetric bone void filler piece may be made of synthetic material such as ceramic, including members of the calcium phosphate family. Specifically, the filler piece may be made of or may comprise tricalcium phosphate, which is biodegradable. The tricalcium phosphate may be of a crystal structure that is either α-tricalcium phosphate or β-tricalcium phosphate or both, in any proportion. For example, the tricalcium phosphate may comprise at least about 80% β-tricalcium phosphate and not more than about 20% α-tricalcium phosphate. Hydroxyapatite is another suitable member of the calcium phosphate family, which is nonresorbable. The axisymmetric bone void filler piece could also be made at least partially of demineralized bone matrix, such as by having particles of demineralized bone matrix joined to each other by a binder substance. In another embodiment, the axisymmetric bone void filler piece may comprise one or more polymers such as a biodegradable polymer.

The bone void filler pieces of the invention may further comprise any of various bioactive materials, such as those described in U.S. patent application Ser. No. 10/122,129, filed Apr. 12, 2002, which is herein incorporated by reference in its entirety.

Bone void filler pieces of the invention may further comprise a radioopaque marker, which may be resorbable.

The bone void filler pieces of the invention may be sterile and may be packaged under sterile conditions.

The invention also includes a method of installing the filler piece such that the dimensions of the bone void filler pieces of the invention closely fit the dimensions of the void in the bone, without substantially altering the as-manufactured surface of the axisymmetric or wedge-shaped bone void filler piece, or without altering the bone void filler pieces of the invention to an extent that obliterates their as-manufactured patterned surface. The method may include cutting of the patient's native bone to an appropriate shape and dimension either by hand or by a powered cutting tool, either with or without a template. In the event that further shaping of the bone void filler piece of the invention is needed before it is installed, material may be removed from the bone void filler pieces of the invention, such as from the large substantially flat surfaces of the filler piece, so as to improve fit with the bone void.

The method of installation may also include, after the bone void filler piece(s) of the invention has been implanted, shaping the exposed portion of the filler piece by removing material from it. This may be desirable, for example, if the surface of a piece which remains exposed as the external surface of the installed bone void filler piece of the invention, has been manufactured as a flat surface but the adjoining bone has curved surfaces, or in general, if the bone void filler piece of the invention is in any way oversized compared to the adjacent bone. For example, the exposed edge of the bone void filler piece of the invention may be shaped by removal of material so as to match contours of native bone nearby. Such reshaping may be helpful in reducing the formation of adhesions between the bone void filler piece of the invention and adjacent soft tissue, which can be painful for the patient.

The method of installation may include soaking the bone void filler piece of the invention in blood, platelet rich plasma, bone marrow, or other bodily fluids prior to final implantation in the patient. Such soaking may help to promote the ingrowth of natural bone.

The void in the bone may be surgically created for purposes of harvesting donor bone. Alternatively, the void in the bone may be surgically created for any other reason.

The invention also includes aspects of methods of manufacture of the bone void filler pieces of the invention. The method may include three dimensional printing (“3DP”). 3DP provides the ability to precisely determine local geometric features and composition of a manufactured piece, to an extent that is not possible with most other manufacturing methods. Because the architecture or structure of the bone void filler pieces of the invention can be controlled through the use of three dimensional printing techniques, namely controlled particle packing with defined interparticle pores, good bone ingrowth is achievable once with optimal appropriate printing parameters. Furthermore, controlled, repeatable resportion characteristics and osteoconductivity are achieved. The bone void filler pieces of the invention eliminates substantial variability in tissue response due to the random distributions in pore size and internal structure.

Other forms of manufacturing, including but not limited to molding, could also be used in the manufacture of the described filler piece. The manufacturing method also may include a chemical reaction to form a desired substance, such as tricalcium phosphate, from precursors. The manufacturing method also may include the use of a decomposable porogen. Any of these aspects of the method may be used either separately or together in any combination.

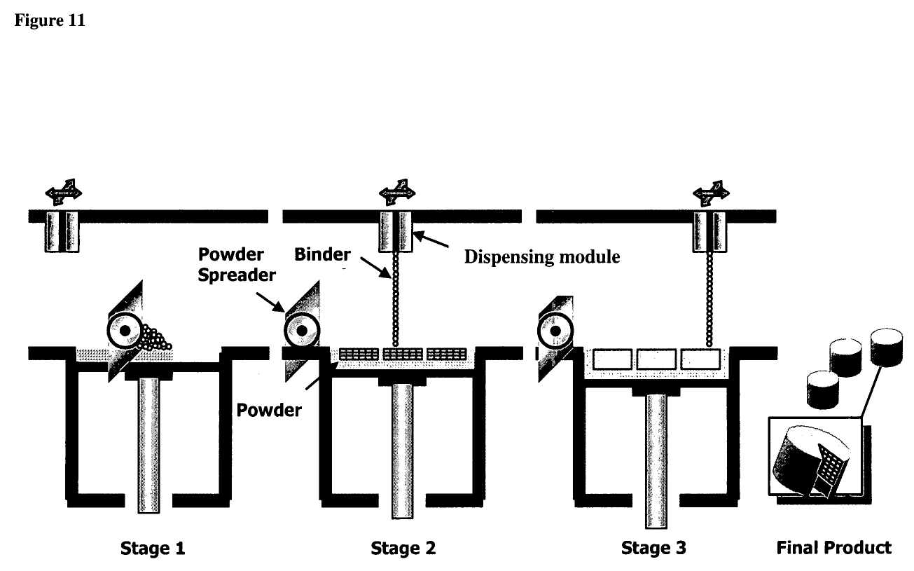

Three dimensional printing, illustrated in FIG. 11, includes a set of steps which may be repeated as many times as are necessary to manufacture a bone void filler piece. Three dimensional printing is described in U.S. Pat. Nos. 5,204,055 and 6,139,574, the disclosures of each of which are herein incorporated by reference in their entireties. At the beginning of the set of steps, powder may be deposited in the form of a layer. The powder may be deposited by roller-spreading or by other means such as slurry deposition.

In one aspect of the present invention, the deposited powder may comprise particles of precursors of a ceramic. Precursors may comprise hydroxyapatite and dicalcium phosphate, and even calcium pyrophosphate or other calcium-phosphorus compounds, as described elsewhere herein or in the incorporated references. The ceramic or precursor may in general include any member or members of the calcium phosphate family.

In another aspect of the invention, the deposited powder may comprise the desired ceramic. For example, the deposited ceramic may be tricalcium phosphate, and, in particular, may be β-tricalcium phosphate. The ceramic may be any other desired ceramic or mixture of ceramics.

In another method of manufacture of the invention, the deposited powder may comprise particles of a porogen, which may be decomposable. The proportion of the porogen to the other particles in the deposited powder may be chosen so as to result in a finished product having a desired porosity. The sizes and size distribution of the other particles (which may include ceramic and/or precursors) and the particles of the porogen may be chosen so as to determine the size and size distribution of the pores in the finished product. The porogen may be lactose, such as spray dried lactose, or another sugar, or in general, any substance which is capable of decomposing, into gaseous decomposition products, at a temperature which is permissible for the materials already in the product at the time of decomposition. This may be done with the other particles comprising either the desired ceramic, or precursors, or both. The average size of the particles of the porogen may be larger than the average size of the particles of the rest of the powder, and may even be significantly larger such as by a factor of approximately 5. For example, the size of the lactose particles may be on average about 120 to about 150 micrometers while the size of the other particles may be on average about 10 micrometers. The proportion of decomposable porogen to other substances may be, for example, about 0 to about 50% by weight. It has been found that a powder containing a combination of lactose and ceramic or precursors is easier to roller-spread than a powder containing only ceramic or precursors without lactose.

In still other aspects of the invention, the deposited powder may be or may include polymer particles or particles of demineralized bone matrix. Particles of demineralized bone matrix may be in an average size range of about 200 micrometers.

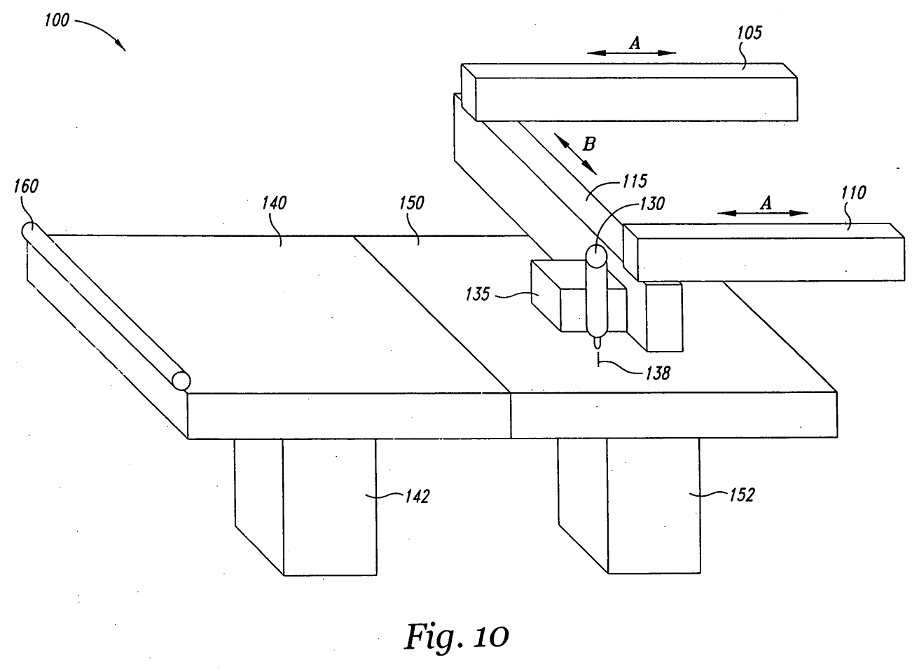

After the deposition of a powder layer, drops of a liquid may be deposited onto the powder layer to bind powder particles to each other and to other bound powder particles. FIG. 11 shows a general schematic of a 3DP manufacturing process, where a powder layer is spread by a powder spreader, followed by dispensing of a binder liquid from a dispensing module. At each powder layer, timing of drop deposition such as from a printhead may be coordinated, for example by software, with the motion of the printhead in two axes, to produce a desired pattern of deposited droplets. FIG. 10 provides a typical three-dimensional printing apparatus [100] in accordance with the prior art. The apparatus [100] includes a roller [160] for rolling powder from a feed bed [140] onto a build bed [150]. Vertical positioners [142 and 152, respectively] position the feed bed [140] and the build bed [150] respectively. Slow axis rails [105, 110] provide support for a printhead [130] in the direction of slow axis motion A, and fast axis rail [115] provides support for the printhead [130] in the direction of fast axis motion B. The printhead [130] is mounted on support [135], and dispenses liquid binder [138] onto the build bed [150] to form the three-dimensional object.

The term droplets will be understood to include not only spherical drops but any of the various possible dispensed fluid shapes or structures as are known in the art. The liquid may be dispensed by a dispensing device suitable for dispensing small quantities of liquid drops, which may resemble an ink-jet printhead. For example, the dispensing device could be a microvalve (The Lee Company, Essex, Conn.) or it could be a piezoelectric drop-on-demand printhead, a continuous-jet printhead, or any other type of printhead as is known in the art. The liquid may comprise a binding substance dissolved in a solvent, which may be water.

The binding substance may be capable of decomposing into gaseous decomposition products at a temperature that is permissible for the materials already in the product at the time of decomposition. The binding substance may, for example, be polyacrylic acid. In certain materials systems (such as demineralized bone matrix), the binder substance may be left in the finished product. In certain materials systems, such as polymers, the binder liquid may be a pure solvent.

After this liquid dispensing process is completed on one layer, another layer of powder may be spread and the liquid dispensing may be repeated, and so on until a complete three-dimensional object has been built. The printing pattern(s) in each printed layer may in general be different from the printing pattern(s) in other layers, with each printing pattern being chosen appropriately so as to form an appropriate portion of a desired piece. During 3DP printing, the unbound powder supports the bound shape and the later deposited layers of powder. At the end of the 3DP printing process the powder particles that are unbound and untrapped may be removed, leaving only the shape which has been bound together.

After separation of the bound shape from unbound powder, the bound shape may be processed with a heat treatment suitable to accomplish any one or more, or all, of several purposes. (For certain powder materials such as polymer and demineralized bone matrix, heat treatment may be impermissible.) The heating may be performed so as to thermally decompose the decomposable porogen (if used) so that the porogen exits the bound shape in the form of gaseous decomposition products. A typical decomposable porogen may decompose at temperatures below 400° C. The heating may also be performed so as to thermally decompose the binder substance so that the binder substance also exits the bound shape in the form of gaseous decomposition products. A typical temperature for this purpose may be about 400° C. If ceramic particles are used, the heating may also be performed so as to partially sinter the ceramic particles together, thereby forming a porous structure of ceramic particles bound directly to other ceramic particles. A typical temperature and duration for this purpose, for members of the calcium phosphate family, may be about 1100° C. to about 1300° C. for about one to about several hours, depending on the ceramic. The heating may also be performed so as to cause the reaction of precursors to form the desired final ceramic, if such materials are used. The described heating may be performed in an oven whose atmosphere is ordinary atmospheric air, or can be performed in another special atmosphere if needed.

The formation of a desired final ceramic from precursors can involve a chemical reaction. For example, hydroxyapatite, which is Ca₁₀(PO₄)₆(OH)₂, plus dicalcium phosphate, which is CaHPO₄, yields tricalcium phosphate, which is Ca₃(PO₄)₂. The following is an exemplary reaction as described above: Ca₅(PO₄)₃OH+CaHPO₄--->2Ca₃(PO₄)₂+H₂O (Hydroxyapatite+Dibasic Calcium Phosphate=Tricalcium Phosphate+Water).

In one embodiment of the invention, the desired ceramic is produced from a specific combination of the following proportions, which involves adding calcium pyrophosphate in an amount of about 3% to the powder. The proportions are as follows: about 58.2% precursors, about 38.8% lactose, and about 3% calcium pyrophosphate. Calcium pyrophosphate is Ca₂P₂O₇. Further details of chemical reaction among calcium-phosphorus compounds are given in commonly assigned U.S. patent application Ser. No. 10/122,129, filed Apr. 12, 2002, the disclosure of which is herein incorporated by reference in its entirety. This reaction may take place at elevated temperatures such as about 1100 C or higher, depending on individual chemistry and time duration. The methods of the present invention may further include the formation of a reaction product, such as a ceramic such as tricalcium phosphate, from precursors, regardless of whether three dimensional printing is or is not used. The methods of the present invention can include the use of a decomposable porogen, regardless of whether three dimensional printing is used. The methods of the present invention can include the use of a decomposable binder substance, regardless of whether three dimensional printing is used.

For certain applications such as simple geometries, the bone void filler piece of the invention could also be manufactured by molding, or other appropriate methods. After any method of manufacturing the bone void filler piece of the invention, it is possible to apply one or more bioactive substances to the bone void filler piece of the invention such as by dispensing or dipping. The invention also includes a bone void filler piece of the invention manufactured by any of the described methods.

The invention also includes tooling and/or templates suitable for insuring that the final dimensions of the void closely match the as-manufactured dimensions of the bone void filler piece of the invention, for example to within a tolerance of about 0.5 millimeters. Either the cutting tool or the template may be coordinated with the pre-manufactured dimensions of the filler piece, either to insure a substantially exact fit or to insure a fit with a known amount of interference.

The tooling may include, for example, a rasp suitable to remove bone by abrading it. The tooling may include sharp-edged cutting tools, either hand-held or tools suitable to be driven by a powered driver, such as a rotary driver. The tooling may be tooling which itself determines the contour of the void, such as if the cutting is done all at once, or tooling which cuts smaller portions over numerous times and determines the contour of the voids as a result of the positions through which the tooling is moved.

The invention also includes a kit containing the filler piece, or more than one filler piece possibly of differing dimensions. The kit may further include one or more appropriate items such as tooling and/or templates suitable for assuring a close fit between the void and the filler piece with minimal modification to the filler piece. The additional item or items in the kit may include any one or more of the following: tools for installation of the bone void filler into the bone void; tools for creation of the bone void; a spreader for spreading apart subcomponents of the bone void filler, if the bone void filler is so designed; putty, paste or adhesive suitable to adhere the bone void filler to the adjacent bone or other tissue; and any other instruments or materials useful during surgery. The tools for creation of the bone void may be geometrically matched to the bone void filler so as to result in a desired fit or a desired gap or even a desired interference between the bone void filler and the bone void created using the tools.

The kit may further include bone putty or other substances that may be useful during surgery. The invention also includes a kit comprising a wafer constructed in accordance with the teachings herein, together with one or more additional items useful with the wafer. The additional item or items in the kit may include any one or more of the following: tools for installation of the wafer into the patient; putty, paste or other suitable material for use in the vicinity of the wafer; and any other instruments or materials useful during surgery.

The invention may also include a tool suitable for pushing on a substantial exposed area of the filler piece after the filler piece has been installed in the void in the patient's bone. Such pushing may be useful for setting the filler piece securely in the void, and also for creating force between the bone and the adjacent bone sufficient to reduce or stop bleeding from the freshly-cut surfaces of the bone, as described elsewhere herein.

Bleeding from freshly cut bone often does not stop immediately after cutting, and such bleeding can obscure viewing of the surgical site and lengthen the surgical time. As discussed supra regarding the wedge-shaped bone void filler, it may be possible to use the bone void filler pieces of the present invention to restrict or stop such bleeding by “stuffing” the void with the filler piece.

The wafer-shaped bone void filler may be used for a variety of medical indications. It may be used for complicated or difficult-to-heal fractures or non-unions in bones. It may also be used in failure-to-knit situations. Any of these situations may result from trauma, from the surgical removal of bone, or for any other reason. The wafer-shaped bone void filler may also be used for lengthening or otherwise adjusting bones.

The invention also includes installing the bone void filler pieces of the invention in a bone void. Installation can include using a bone putty, adhesive, or other such substance to retain the bone void filler piece in place, and to help fill gaps. Such materials are commonly used in the field of orthopedics to fill voids in bone fractures and surgery. Such a material may be placed between the bone void filler piece of the invention and the bone void. Such a material may be either natural or synthetic in origin.

Installation can include forcing or tapping the bone void filler piece into place to create frictional fit within the bone void. Installation may also include forcing or tapping the bone void filler into place so as to crush or shear off certain features of the bone void filler, thereby creating a frictional restraint. This can be done with the bone void filler pieces of the invention which are either untapered or tapered. The use of insertion force resulting in possible localized crushing may be useful in helping to limit the flow of blood which often takes place from freshly cut bone.

The bone void filler pieces of the present invention can be used for any of a variety of medical indications. The bone void filler pieces of the present invention can be used to fill cylindrical defects, such as those left by a drill bit. They can be used to fill voids such as cylindrical or other axisymmetric voids made in the iliac crest to harvest bone graft. They can be used to fill cylindrical or other axisymmetric voids made in the femur and tibia (or other bones) during ligament reconstruction. They can be used to fill defects following removal of a cylindrical implant (e.g. a sliding hip screw). They can be used to fill voids when returning to graft a cylindrical or other axisymmetric defect after treatment of an infection. The described bone void filler pieces can also be used by a surgeon after performing a core decompression drilling of the femoral neck or any other bone for osteonecrosis.

The bone void filler pieces may be used for treatment of a variety of medical indications including situations that may result from the donation of bone, from trauma, from any surgical removal of bone, or for any other reason.

In general, surface recesses or channels can be on any surface of the filler pieces. The bone void filler pieces of the invention provide the benefits of ceramic as a material or provide the benefit of demineralized bone matrix as a material, while also providing a desired pre-manufactured shape. The invention also provides features such as surface recesses or channels, which are believed to promote the ingrowth of natural bone. The invention can provide for the restriction or stoppage of the flow of blood from freshly cut bone.

The above description of illustrated embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. Aspects of the invention can be modified, if necessary, to employ the process, apparatuses and concepts of the various patents and applications described above to provide yet further embodiments of the invention. The various embodiments described above can be combined to provide further embodiments. These and other changes can be made to the invention in light of the above detailed description. In general, in the following claims, the terms used should not be construed to limit the invention to the specific embodiments disclosed in the specification and the claims, but should be construed to include all bone substitutes that operate under the claims. Accordingly, the invention is not limited by the disclosure, but instead the scope of the invention is to be determined entirely by the following claims.

From the foregoing it will be appreciated that, although specific embodiments of the invention have been described herein for purposes of illustration, various modifications may be made without deviating from the spirit and scope of the invention. Accordingly, the invention is not limited except as by the appended claims.

Certain shaped bone void fillers and compositions, as well as methods of manufacturing the same were disclosed in U.S. Provisional Application No. 60/512,498, filed Oct. 17, 2003; U.S. Provisional Application No. 60/512,414, filed Oct. 17, 2003; U.S. Provisional Application No. 60/577,736, filed Jun. 7, 2004; and U.S. patent application Ser. No. 10/122,129, filed Apr. 12, 2002, the disclosures of each of which are herein incorporated by reference in their entireties.

Certain methods, systems and apparatuses for use in three-dimensional printing and for engineered regenerative biostructures were disclosed in U.S. patent application Ser. No. 10/122,129, filed Apr. 12, 2002; U.S. patent application Ser. No. 10/189,795, filed Jul. 3, 2002; U.S. patent application Ser. No. 10/190,333, filed Jul. 3, 2002; U.S. patent application Ser. No. 10/189,799, filed Jul. 3, 2002; U.S. patent application Ser. No. 10/189,166, filed Jul. 3, 2002; U.S. patent application Ser. No. 10/189,153, filed Jul. 3, 2002; and U.S. patent application Ser. No. 10/189,797, filed Jul. 3, 2002, the disclosures of each of which are herein incorporated by reference in their entireties.

1. A porous osteoconductive bone void filler piece comprising a matrix of interconnected particles comprising controlled particle packing providing controlled inter-particle pores, wherein said bone void filler piece comprises bone-contacting surfaces which are substantially opposed to each other and have surface tangents which are angled with respect to each other at an angle of less than about 30 degrees.

2. The bone void filler piece of claim 1, further comprising demineralized bone matrix.

3. The bone void filler piece of claim 1, wherein the interconnected particles comprise a ceramic which is a member of the calcium phosphate family.

4. The bone void filler piece of claim 3, wherein the calcium phosphate family member is tricalcium phosphate.

5. The bone void filler piece of claim 1, wherein the filler piece has pores which are suitable to wick bodily fluids.

6. The bone void filler piece of claim 1, wherein the filler piece has pores which make up between approximately 40% and approximately 70% by volume of the bone void filler.

7. The bone void filler piece of claim 1, wherein the filler piece comprises pores having an average pore size of about 60 micrometers.

8. The bone void filler piece of claim 1, wherein the filler piece comprises pores which range from approximately 1 micrometer to approximately 300 micrometers.

9. The bone void filler piece of claim 1, wherein the filler piece has recessed surface features or channels therethrough.

10. The bone void filler piece of claim 9, wherein the recessed surface features or channels have cross-dimensions in the range of about 50 micrometers to about 3000 micrometers.

11. The bone void filler piece of claim 10, wherein the recessed surface features or channels are positioned such that each point in the filler is within at most about 2 millimeters from a surface feature or a channel.

12. The bone void filler piece of claim 1, wherein the filler piece has the shape of a wedge or a truncated wedge defined by a first substantially flat surface and a second substantially flat surface which is angled with respect to the first substantially flat surface.

13. The bone void filler piece of claim 12, wherein the filler piece has channels extending from the first substantially flat surface to the second substantially flat surface.

14. The bone void filler piece of claim 1, wherein the filler piece has recessed surface features in at least some bone-contacting surface.

15. The bone void filler piece of claim 1, wherein the filler piece has ribs or high spots.

16. The bone void filler piece of claim 1, wherein the filler piece has a carrying feature suitable to allow the filler piece to be gripped or carried by a carrying tool.

17. The bone void filler piece of claim 1, further comprising at least one bioactive substance.

18. The bone void filler piece of claim 1, further comprising a radioopaque marker.

19. A porous osteoconductive bone void filler comprising pores having an average pore size of about 60 micrometers, and wherein said bone void filler comprises bone-contacting surfaces which are substantially opposed to each other and have surface tangents which are angled with respect to each other at an angle of less than about 30 degrees.

20. The bone void filler piece of claim 19, wherein the filler piece has a shape of a wedge or a truncated wedge defined by a first substantially flat surface and a second substantially flat surface which is angled with respect to the first substantially flat surface, and wherein the filler channels has channels extending from the first substantially flat surface to the second substantially flat surface.

21. The bone void filler piece of claim 19, wherein bone-contacting surfaces are crushable, and having an exposed surface suitable to have force exerted on it for pushing the filler piece into the bone void.

22. The bone void filler piece of claim 19, further comprising demineralized bone matrix.

23. The bone void filler piece of claim 19, wherein the filler piece has pores which are suitable to wick bodily fluids.

24. The bone void filler piece of claim 19, wherein the filler piece has pores which make up between approximately 40% and approximately 70% by volume of the bone void filler.

25. The bone void filler piece of claim 19, wherein the filler piece comprises pores which range from approximately 1 micrometer to approximately 300 micrometers.

26. The bone void filler piece of claim 19, wherein the filler piece has recessed surface features or channels therethrough.

27. The bone void filler piece of claim 26, wherein the recessed surface features or channels have cross-dimensions in the range of about 50 micrometers to about 3000 micrometers.

28. The bone void filler piece of claim 27, wherein the recessed surface features or channels are positioned such that each point in the filler is within at least about 2 millimeters from a surface feature or a channel.

29. The bone void filler piece of claim 19, wherein the filler piece has the shape of a wedge or a truncated wedge defined by a first substantially flat surface and a second substantially flat surface which is angled with respect to the first substantially flat surface.

30. The bone void filler piece of claim 29, wherein the filler piece has channels extending from the first substantially flat surface to the second substantially flat surface.

31. The bone void filler piece of claim 19, wherein the filler piece has recessed surface features in at least some bone-contacting surface.

32. The bone void filler piece of claim 19, wherein the filler piece has ribs or high spots.

33. The bone void filler piece of claim 19, wherein the filler piece has a carrying feature suitable to allow the filler piece to be gripped or carried by a carrying tool.

34. The bone void filler piece of claim 19, further comprising at least one bioactive substance.

35. The bone void filler piece of claim 19, further comprising a radioopaque marker.

36. A porous osteoconductive bone void filler piece comprising a matrix of interconnected particles comprising controlled particle packing providing controlled inter-particle pores and further comprising a member of the calcium phosphate family, wherein said bone void filler piece comprises a wafer shape suitable to be implanted between the surfaces of bones.

37. The bone void filler piece of claim 36, further comprising demineralized bone matrix.

38. The bone void filler piece of claim 36, wherein the calcium phosphate family member is tricalcium phosphate.

39. The bone void filler piece of claim 36, wherein the filler piece has pores which are suitable to wick bodily fluids.

40. The bone void filler piece of claim 36, wherein the filler piece has pores which make up between approximately 40% and approximately 70% by volume of the bone void filler.

41. The bone void filler piece of claim 36, wherein the wafer has pores which have a pore size distribution having a peak between about 8 micrometers and about 20 micrometers.

42. The bone void filler piece of claim 36, wherein the filler piece comprises pores which range from about 1 micrometer to about 300 micrometers.

43. The bone void filler piece of claim 36, wherein the filler piece has recessed surface features or channels therethrough.

44. The bone void filler piece of claim 43, wherein the recessed surface features or channels have a smallest dimension along a surface of the wafer, which is in the range from approximately 50 micrometers to approximately 500 micrometers.

45. The bone void filler piece of claim 36, wherein the filler piece has recessed surface features in at least some bone-contacting surface.

46. The bone void filler piece of claim 36, wherein the filler piece has ribs or high spots.

47. The bone void filler piece of claim 36, wherein the filler piece has a carrying feature suitable to allow the filler piece to be gripped or carried by a carrying tool.

48. The bone void filler piece of claim 36, further comprising at least one bioactive substance.

49. A porous osteoconductive bone void filler piece comprising a matrix of interconnected particles comprising controlled particle packing providing controlled inter-particle pores and further comprising a member of the calcium phosphate family, wherein said bone void filler piece comprises a wafer shape having an average pore size of about 60 micrometers.

50. The bone void filler piece of claim 49, further comprising demineralized bone matrix.

51. The bone void filler piece of claim 49, wherein the calcium phosphate family member is tricalcium phosphate.

52. The bone void filler piece of claim 49, wherein the filler piece has pores which are suitable to wick bodily fluids.

53. The bone void filler piece of claim 49, wherein the filler piece has pores which make up between approximately 40% and approximately 70% by volume of the bone void filler.

54. The bone void filler piece of claim 49, wherein the filler piece comprises pores which range from approximately 1 micrometer to approximately 300 micrometers.

55. The bone void filler piece of claim 49, wherein the filler piece has recessed surface features or channels therethrough.

56. The bone void filler piece of claim 55, wherein the recessed surface features or channels have a smallest dimension along a surface of the wafer, which is in the range from approximately 50 micrometers to approximately 500 micrometers.

57. The bone void filler piece of claim 49, wherein the filler piece has recessed surface features in at least some bone-contacting surface.

58. The bone void filler piece of claim 49, wherein the filler piece has ribs or high spots.

59. The bone void filler piece of claim 49, wherein the filler piece has a carrying feature suitable to allow the filler piece to be gripped or carried by a carrying tool.

60. The bone void filler piece of claim 49, further comprising at least one bioactive substance.

61. A porous osteoconductive bone void filler piece comprising a matrix of interconnected particles comprising controlled particle packing providing controlled inter-particle pores and further comprising a member of the calcium phosphate family, wherein said bone void filler piece comprises an axisymmetric overall shape suitable to be implanted into and fit closely in the bone void.

62. The bone void filler piece of claim 61, further comprising demineralized bone matrix.

63. The bone void filler piece of claim 61, wherein the calcium phosphate family member is tricalcium phosphate.

64. The bone void filler piece of claim 61, wherein the filler piece has pores which are suitable to wick bodily fluids.

65. The bone void filler piece of claim 61, wherein the filler piece has pores which make up between approximately 20% and approximately 50% by volume of the bone void filler.

66. The bone void filler piece of claim 61, wherein the filler piece has pores which make up between approximately 40% and approximately 70% by volume of the bone void filler.

67. The bone void filler piece of claim 61, wherein the wafer has pores which have a pore size distribution between about 8 micrometers and about 20 micrometers.

68. The bone void filler piece of claim 61, wherein the filler piece comprises pores having an average pore size between about 60 micrometers and about 90 micrometers.

69. The bone void filler piece of claim 61, wherein the filler piece comprises pores which range from about 7 micrometers to about 1000 micrometers.

70. The bone void filler piece of claim 61, wherein the filler piece has recessed surface features or channels therethrough.

71. The bone void filler piece of claim 61, wherein the filler piece has recessed surface features in at least some bone-contacting surface.

72. The bone void filler piece of claim 61, wherein the filler piece has ribs or high spots.

73. The bone void filler piece of claim 61, wherein the filler piece has a carrying feature suitable to allow the filler piece to be gripped or carried by a carrying tool.

74. The bone void filler piece of claim 61, further comprising at least one bioactive substance.

75. The bone void filler piece of claim 61, wherein the filler piece is suitable to be inserted into the bone void with a translational motion.

76. The bone void filler piece of claim 61, wherein the filler piece is suitable to be inserted into the bone void with simultaneous translational and rotational motion.

77. The bone void filler piece of claim 61, wherein the filler piece is suitable to be inserted into the bone void with translational motion followed by rotational motion.

78. The bone void filler piece of claim 61, wherein the filler piece fits into the bone void with a frictional fit.

79. The bone void filler piece of claim 61, wherein the filler piece has a taper on at least one external surfaces suitable to frictionally engage the bone void.

80. The bone void filler piece of claim 61, wherein the filler piece has ribs or high spots.

81. The bone void filler piece of claim 80, wherein said ribs or high spots are crushable.

82. The bone void filler piece of claim 61, wherein the filler piece is suitable to be broken into at least two parts which together may be inserted into and fit closely in the bone void.

83. The bone void filler piece of claim 61, wherein the filler piece has a chamfer or taper on at least one external surface suitable to guide the bone void filler into the bone void.

84. The bone void filler piece of claim 61, wherein the filler piece has at least one carrying feature suitable to allow the bone void filler to be gripped or carried by a tool.

85. The bone void filler piece of claim 61, further comprising at least one bioactive substance.

86. The bone void filler piece of claim 70, wherein the surface recesses or channels have a smallest dimension along a surface of the filler piece, which is in the range from approximately 500 micrometers to approximately 3000 micrometers.

87. The bone void filler piece of claim 61, wherein the generally axisymmetric shape of the filler piece comprises an overall axis of symmetry, further comprising at one end a generally axisymmetric transition region having a transition axis of symmetry which substantially coincides with the overall axis of symmetry.

88. The bone void filler piece of claim 87, wherein the filler piece is substantially cylindrical.

89. The bone void filler piece of claim 87, wherein the filler piece is substantially frusto-conical.

90. The bone void filler piece of claim 87, wherein the transition region comprises a chamfer.

91. The bone void filler piece of claim 87, wherein the transition region comprises a smooth curve.

92. A method of manufacturing a bone void filler piece, comprising depositing a layer of powder comprising powder particles, depositing onto the layer of powder in selected positions on the powder layer a binder liquid suitable to bind powder particles to other powder particles, and repeating the above steps to form a bound filler piece having bone-contacting surfaces which are substantially opposed to each other and having surface tangents which are angled with respect to each other at an angle of less than approximately 30 degrees, and separating the bound shape from unbound powder.

93. The method of claim 92, wherein the filler piece further comprises surface recesses or internal channels engineered into the piece through selective deposition of the binder liquid.

94. The method of claim 92, wherein the powder particles comprise at least one member of the calcium phosphate family.

95. The method of claim 94, wherein the calcium phosphate family member comprises tricalcium phosphate.

96. The method of claim 92, wherein the powder particles comprise a mean size in the range of about 20 micrometers to about 40 micrometers.

97. The method of claim 94, further comprising heating the bound shape to a suitable temperature to partially sinter the bound shape following separation of the bound shape from the unbound powder.

98. The method of claim 92, wherein depositing the powder particles comprises depositing powder particles which have a mean size in the range of about 5 micrometers to about 50 micrometers.

99. The method of claim 92, wherein the powder particles comprise precursors of at least one member of the calcium phosphate family.

100. The method of claim 99, further comprising heating the bound shape to a temperature sufficient to cause the precursors to react to form a desired ceramic.

101. The method of claim 100, wherein the precursors react to form a desired ceramic at a temperature between about 1100 C and about 1300 C.

102. The method of claim 99, wherein the precursors comprise hydroxyapatite and dicalcium phosphate.

103. The method of claim 92, wherein depositing the powder particles comprises depositing powder particles which further comprise a decomposable porogen.

104. The method of claim 99, wherein depositing the powder particles comprises depositing powder particles which further comprise a decomposable porogen.

105. The method of claim 104, wherein the decomposable porogen comprises lactose or another sugar.

106. The method of claim 104, wherein the particles of the precursors have a first average particle size, and the particles of the decomposable porogen have a second average particle size, and the second average particle size is at least approximately 5 times as large as the first particle size.

107. The method of claim 106, wherein the bound piece is heated to a temperature sufficient to thermally decompose the binder substance into gaseous decomposition products.

108. The method of claim 92, wherein the powder particles comprise particles of demineralized bone matrix.

109. The method of claim 108, wherein the deposited powder particles have a mean size in the range of about 200 micrometers.

110. A method of manufacturing a bone void filler piece, comprising depositing a layer of powder comprising powder particles, depositing onto the layer of powder in selected positions on the powder layer a binder liquid suitable to bind powder particles to other powder particles, and repeating the above steps to form a shape comprising an axisymmetric overall shape suitable to be implanted into and fit closely in a bone void, and separating the bound shape from unbound powder.

111. The method of claim 110, wherein the filler piece further comprises surface recesses or internal channels engineered into the piece through selective deposition of the binder liquid.

112. The method of claim 110, wherein the powder particles comprise at least one member of the calcium phosphate family.

113. The method of claim 112, wherein the calcium phosphate family member comprises tricalcium phosphate.

114. The method of claim 110, wherein the powder particles comprise a mean size in the range of about 10 micrometers.

115. The method of claim 112, further comprising heating the bound shape to a suitable temperature to partially sinter the bound shape following separation of the bound shape from the unbound powder.

116. The method of claim 110, wherein depositing the powder particles comprises depositing powder particles which have a mean size in the range of about 13 micrometers to about 23 micrometers.

117. The method of claim 110, wherein the powder particles comprise precursors of at least one member of the calcium phosphate family.

118. The method of claim 117, further comprising heating the bound shape to a temperature sufficient to cause the precursors to react to form a desired ceramic.

119. The method of claim 118, wherein the precursors react to form a desired ceramic at a temperature between about 1100 C and about 1300 C.

120. The method of claim 117, wherein the precursors comprise hydroxyapatite and dicalcium phosphate.

121. The method of claim 110, wherein depositing the powder particles comprises depositing powder particles which further comprise a decomposable porogen.

122. The method of claim 117, wherein depositing the powder particles comprises depositing powder particles which further comprise a decomposable porogen.

123. The method of claim 122, wherein the decomposable porogen comprises lactose or another sugar.

124. The method of claim 122, wherein the particles of the precursors have a first average particle size, and the particles of the decomposable porogen have a second average particle size, and the second average particle size is at least approximately 5 times as large as the first particle size.

125. The method of claim 124, wherein the bound piece is heated to a temperature sufficient to thermally decompose the binder substance into gaseous decomposition products.

126. The method of claim 110, wherein the powder particles comprise particles of demineralized bone matrix.

127. The method of claim 126, wherein the deposited powder particles have a mean size in the range of about 200 micrometers.

128. A method of manufacturing a bone void filler piece, comprising depositing a layer of powder comprising powder particles, depositing onto the layer of powder in selected positions on the powder layer a binder liquid suitable to bind powder particles to other powder particles, and repeating the above steps to form a shape which is a wafer having surface recesses or internal channels, and separating the bound shape from unbound powder.

129. The method of claim 128, wherein the filler piece further comprises surface recesses or internal channels engineered into the piece through selective deposition of the binder liquid.

130. The method of claim 128, wherein the powder particles comprise at least one member of the calcium phosphate family.

131. The method of claim 130, wherein the calcium phosphate family member comprises tricalcium phosphate.

132. The method of claim 128, wherein the powder particles comprise a mean size in the range of about 20 micrometers to about 40 micrometers.

133. The method of claim 130, further comprising heating the bound shape to a suitable temperature to partially sinter the bound shape following separation of the bound shape from the unbound powder.

134. The method of claim 128, wherein depositing the powder particles comprises depositing powder particles which have a mean size in the range of about 5 micrometers to about 50 micrometers.

135. The method of claim 128, wherein the powder particles comprise precursors of at least one member of the calcium phosphate family.

136. The method of claim 135, further comprising heating the bound shape to a temperature sufficient to cause the precursors to react to form a desired ceramic.

137. The method of claim 136, wherein the precursors react to form a desired ceramic at a temperature between about 1100 C and about 1300 C.

138. The method of claim 135, wherein the precursors comprise hydroxyapatite and dicalcium phosphate.

139. The method of claim 128, wherein depositing the powder particles comprises depositing powder particles which further comprise a decomposable porogen.

140. The method of claim 135, wherein depositing the powder particles comprises depositing powder particles which further comprise a decomposable porogen.

141. The method of claim 140, wherein the decomposable porogen comprises lactose or another sugar.

142. The method of claim 140, wherein the particles of the precursors have a first average particle size, and the particles of the decomposable porogen have a second average particle size, and the second average particle size is at least approximately 5 times as large as the first particle size.

143. The method of claim 142, wherein the bound piece is heated to a temperature sufficient to thermally decompose the binder substance into gaseous decomposition products.

144. The method of claim 128, wherein the powder particles comprise particles of demineralized bone matrix.

145. The method of claim 144, wherein the deposited powder particles have a mean size in the range of about 200 micrometers.

146. A kit comprising the bone void filler piece of any one of claims 1, 19, 36, 49 or 61, and at least one item selected from the group consisting of tooling suitable for installing the filler piece into a patient; other surgical instruments; a putty, paste or other material suitable for use near the filler piece or to adhere the bone void filler to adjacent bone; and any combination thereof.

147. A kit according to claim 146, wherein the tooling is dimensionally matched to the bone void filler so as to create a desired fit or a desired gap or a desired geometric interference.

148. The method of any one of claims 99, 117 or 135, wherein the powder particles comprise a composition in the proportions of about 58.2% precursors, about 38.8% lactose, and about 3% calcium pyrophosphate.
